(19) United States
(12) Patent Application Publication
(10) Pub. No.: US 2010/0063258 A1
(43) Pub. Date: Mar. 11, 2010

(54) FUSION PROTEIN CONSTRUCTS

(75) Inventors: James Robert Swartz, Menlo Park, CA (US); Junhao Yang, Palo Alto, CA (US); Alexei M. Voloshin, Newark, CA (US)

Correspondence Address: Stanford University Office of Technology Licensing;Bozicevic, Field & Francis LLP, 1900 University Avenue, Suite 200, East Palo Alto, CA, 94303, US

(73) Assignee: THE BOARD OF TRUSTEES OF THE LELAND STANFORD JUNIOR UNIVERSITY, Palo Alto, CA (US)

(21) Appl. No.: 12/305,614

(22) PCT Filed: Jun. 28, 2007
(86) PCT No.: PCT/US2007/015217

§ 371 Date: Nov. 13, 2009

Related U.S. Application Data
(60) Provisional application No. 60/817,443, filed on Jun. 28, 2006.

(51) Int. Cl. C07K 16/00

(52) U.S. Cl. 530/387.3

(57) ABSTRACT

Polypeptide linkers with defined tertiary structures, usually of defined alpha helical structure, are used to join two domains in a fusion protein. In one embodiment of the invention, a method is provided for the cell-free synthesis of the fusion protein.

### BACKGROUND OF THE INVENTION

Many cellular processes involve proteins with multiple domains. This modular nature of proteins provides many advantages, providing increased stability and new cooperative functions. In addition, chimeric proteins that provide for new functional combinations can be designed from domain modules of different proteins.

The amino acid linkers that join domains can play an important role in the structure and function of multi-domain proteins. There are numerous examples of proteins whose catalytic activity requires proper linker composition. In general, altering the length of linkers connecting domains has been shown to affect protein stability, folding rates and domain-domain orientation (see George and Heringa (2003) Prot. Eng. 15:871-879).

Many studies of natural linker peptides in various protein families have come to the conclusion that linkers lack regular secondary structure, they display varying degrees of flexibility to match their particular biological purpose and are rich in Ala, Pro and charged residues. For example, in a study by Argos (1990) J. Mol. Biol. 211:943-958) it was concluded that preferred linker amino acids are mostly hydrophilic, often polar and usually small. The majority of the linker residues are in coil or bend structures with a mean length of 6.5 residues, and an average flexibility when compared to other protein regions. Differing structures pointed to the importance of the amino acid order to achieve an extended and conformationally stable linker.

Escherichia coil is a widely used organism for the expression of heterologous proteins. It easily grows to a high cell density on inexpensive substrates to provide excellent volumetric and economic productivities. Well established genetic techniques and various expression vectors further justify the use of Escherichia coli as a production host. However, a high rate of protein synthesis is necessary, but by no means sufficient, for the efficient production of active biomolecules. In order to be biologically active, the polypeptide chain has to fold into the correct native three-dimensional structure, including the appropriate formation of disulfide bonds.

In many cases, the recombinant polypeptides have been found to be sequestered within large refractile aggregates known as inclusion bodies. Active proteins can be recovered from inclusion bodies through a cycle of denaturant-induced solubilization of the aggregates followed by removal of the denaturant under conditions that favor refolding. But although the formation of inclusion bodies can sometimes ease the purification of expressed proteins; in most occasions, refolding of the aggregated proteins remains a challenge.

Studies in vitro have demonstrated that, for the vast majority of polypeptides, folding is a spontaneous process directed by the amino acid sequence and the solvent conditions. Yet, even though the native state is thermodynamically favored, the time-scale for folding can vary from milliseconds to days. Kinetic barriers are introduced, for example, by the need for alignment of subunits and sub-domains. And particularly with eukaryotic proteins, covalent reactions must take place for the correctly folded protein to form. The latter types of reaction include disulfide bond formation, cis/trans isomerization of the polypeptide chain around proline peptide bonds, preprotein processing and the ligation of prosthetic groups. These kinetic limitations result in the accumulation of partially folded intermediates, which contain exposed hydrophobic ‘sticky’ surfaces, which promote self-association and formation of aggregates.

For several decades, in vitro protein synthesis has served as an effective tool for lab-scale expression of cloned or synthesized genetic materials. In recent years, in vitro protein synthesis has been considered as an alternative to conventional recombinant DNA technology, because of disadvantages associated with cellular expression. In vivo, proteins can be degraded or modified by several enzymes synthesized with the growth of the cell, and, after synthesis, may be modified by post-translational processing, such as glycosylation, deamidation or oxidation. In addition, many products inhibit metabolic processes and their synthesis must compete with other cellular processes required to reproduce the cell and to protect its genetic information.

Because it is essentially free from cellular regulation of gene expression and does not require the maintenance of cell viability, in vitro protein synthesis has advantages in the production of cytotoxic, unstable, or insoluble proteins. The over-production of protein beyond a predetermined concentration can be difficult to obtain in vivo, because the expression levels are regulated by the concentration of product. The concentration of protein accumulated in the cell generally affects the viability of the cell, so that over-production of the desired protein is difficult to obtain. In an isolation and purification process, many kinds of protein are insoluble or unstable, and are either degraded by intracellular proteases or aggregate in inclusion bodies, so that the loss rate is high.

In vitro synthesis circumvents many of these problems (see Kim and Swartz (1999) Biotechnol. Bioeng. 66:180-188; and Kim and Swartz (2000) Biotechnol. Prog. 16:385-390). Also, through simultaneous and rapid expression of various proteins in a multiplexed configuration, this technology can provide a valuable tool for development of combinatorial arrays for research, and for screening of proteins. In addition, various kinds of unnatural amino acids can be efficiently incorporated into proteins for specific purposes (Noren et al. (1989) Science 244:182-188).

Unlike in vivo gene expression, cell-free protein synthesis uses isolated translational machinery instead of entire cells. As a result, this method eliminates the requirement to maintain cell viability and allows direct control of various parameters to optimize the synthesis/folding of target proteins. Of particular interest is the synthesis of multi-domain proteins. The present invention provides linkers that are useful in these systems.

### Relevant Literature

U.S. Pat. No. 6,337,191 B1; Swartz et al. U.S. Patent Published Application 20040209321; Swartz et al. International Published Application WO 2004/016778; Swartz et al. U.S. Patent Published Application 2005-0054032-A1; Swartz et al. U.S. Patent Published Application 2005-0054044-A1; Swartz et al. International Published Application WO 2005/052117. Calhoun and Swartz (2005) Biotechnol Bioeng 90(5):606-13; Jewett and Swartz (2004) Biotechnol Bioeng 86(1):19-26; Jewett et al. (2002) Prokaryotic Systems for In Vitro Expression. In: Weiner M, Lu Q, editors. Gene cloning and expression technologies. Westborough, Mass.: Eaton Publishing. p 391-411; Lin et al. (2005) Biotechnol Bioeng 89(2):148-56; Liu et al., 2005 Biotechnol Prog 21:460-465; Jewett M C and Swartz J R, 2004 Biotechnol Prog 20:102-109; Zawada and Swath Biotechnol Bioeng, 2006. 94(4): p. 618-24.

The Im9 protein sequence is deposited at Genbank, accession number CAA33863. The structure of the protein is disclosed by Ferguson et al. (1999) J. Mol. Biol. 286:1597-1608.

### SUMMARY OF THE INVENTION

Fusion polypeptides, nucleic acids encoding the fusion polypeptides, and methods of synthesis thereof are provided. In the fusion proteins of the invention, a first polypeptide and a second polypeptide are joined through a linker with defined tertiary structure, usually with defined alpha helical structure. The linker is heterologous to the first polypeptide and second polypeptide components. Linkers of the invention, when inserted between two heterologous polypeptides, unexpectedly provide for an overall higher synthetic yield of full-length, soluble fusion protein, e.g. in cell-free synthesis reactions, as compared to the synthesis of a comparable protein lacking such a linker. Linkers of the invention, when inserted between two heterologous polypeptides, may also unexpectedly provide for increased stability of the fusion protein with respect to proteolytic degradation, as compared to the synthesis of a comparable fusion protein lacking such a linker.

Suitable linker sequences include, without limitation, bacterial immunity proteins or variants thereof, e.g. E. coli Im5, Im6, Im7, Im9, ImmE8, immHu194; and the like, including variants having at least 95% sequence identity to the provided bacterial immunity protein sequences.

In some embodiments of the invention, a method is provided for the cell-free synthesis of a fusion protein, where the fusion protein comprises a first polypeptide and a second polypeptide joined through a heterologous linker of defined tertiary structure to form a fusion protein. In some embodiments the fusion protein comprises one or more domains of a mammalian immunoglobulin proteins, cytokines, etc., e.g. a single chain antibody, constant region domains from heavy and/or light chains, variable region domains, etc.

### BRIEF DESCRIPTION OF THE DRAWINGS

FIG. 1, Protein yield of an immunoglobulin construct with or without the Im9 linker. 1, GM-VL-VH; 2, GM-Im9-VL-VH.

FIG. 2 is an autoradiogram of purified immunoglobulin constructs with and without a bacterial immunity protein linker.

### DETAILED DESCRIPTION OF THE EMBODIMENTS

Compositions and methods are provided for improved synthesis of multi-domain fusion proteins, particularly the cell-free synthesis of such fusion proteins. Polypeptide sequences that provide for fast folding domains are used as linkers to join a first and a second polypeptide in a fusion protein. These linkers of defined tertiary structure are found to provide for increased synthetic yield and product stability when compared to fusion proteins comprising conventional linkers, or in the absence of linkers. Bacterial immunity proteins have been found to be suitable linkers for this purpose. The linker may be used alone or in combination with an additional flexible linker sequence, and may also comprise a tag for purification.

The objects of this invention are accomplished by providing novel polypeptides comprising a first polypeptide and a second polypeptide, separated by a linker polypeptide. DNA encoding the polypeptides and methods for making the polypeptides are also provided. The fusion proteins of this invention can be made by transforming host cells with nucleic acid encoding the fusion, culturing the host cell and recovering the fusion from the culture, or alternatively by generating a nucleic acid construct encoding the fusion and producing the polypeptide by cell free synthesis, which synthesis may include coupled transcription and translation reactions. Also provided are vectors and polynucleotides encoding the fusion protein. In one embodiment of the invention, a method is provided for the cell-free synthesis of a fusion protein, where the fusion protein comprises a polypeptide linker of the present invention.

In cell free synthetic reactions, the use of a linker of the invention provides for greater synthetic yield of intact protein, where intact protein may be measured by various methods, including PAGE, capillary electrophoresis, affinity analysis, functional analysis of protein activity, and the like, as known in the art. The use of the linker provides at least a 20% improvement in the yield of the intact fusion protein as compared to a fusion protein lacking the linker, and may provide for at least a 30%, at least a 40%, at least a 50%, at least a 75%, at least 100% or more improvement in yield.

The fusion proteins may be purified and formulated in pharmacologically acceptable vehicles for administration to a patient. In one embodiment of the invention the fusion protein comprises at least one domain of an immunoglobulin, e.g. a variable region domain; a constant region domain; a single chain Fv fragment; etc. Such fusion proteins find use as immunologically specific reagents; e.g. to increase the plasma half-life of a polypeptide of interest or to target the protein to a particular cell type. In other embodiments the fusion protein contains at least one cytokine domain.

### Linker Polypeptides

A first polypeptide and a second polypeptide are joined through a linker of defined tertiary structure, particularly of defined alpha helical structure, to form a fusion protein. As used herein, the terms “fusion protein” or “fusion polypeptide” or grammatical equivalents herein are meant to denote a protein composed of a plurality of protein components, which are typically unjoined in their native state but are joined by their respective amino and carboxyl termini through a linker of defined tertiary structure to form a single continuous polypeptide. “Protein” in this context includes proteins, polypeptides and peptides. Plurality in this context means at least two, and preferred embodiments generally utilize a first and a second polypeptide joined through a linker.

Linkers of the invention are typically able to fold into a thermodynamically stable structure with reaction durations typically shorter than about 10 seconds as determined by optimized in vitro refolding reactions; and are generally comprised of multiple alpha helices, usually at least about two, at least about three, at least about 4 alpha helices. Preferred linkers are at least about 45 amino acids in length, more usually at least about 55 amino acids in length and not more than about 100 amino acids in length, not more than about 95 amino acids in length, or not more than about 90 amino acids in length. Methods for prediction of folding rates may be found, inter alia, in Debe and Goddard (1999) J Mol Biol. 294(3):619-25, herein specifically incorporated by reference.

The presence of alpha helices in a sequence can be empirically determined, e.g. by CD spectra, where a polypeptide retains CD spectra characteristic of an alpha helix, and where the characteristic spectra persists in the presence of up to 2 M urea. Methods relating to spectral analysis of tertiary structures in polypeptides may be found, inter alia, in Turner et al. J Phys Chem B. 2007 Feb. 22; 111(7):1834; Shepherd et al. J Am Chem Soc. 2005 Mar. 9; 127(9):2974-83; Thulstrup et al. Biopolymers. 2005 May; 78(1):46-52; Jeong et al. Mol Cells. 2004 Feb. 29; 17(1):62-6; Maiti et al. J Am Chem Soc. 2004 Mar. 3; 126(8):2399-408; Maeda et al. J Pept Sci. 2003 February; 9(2):106-13; Verzola et al. Electrophoresis. 2003 March; 24(5):794-800; Wallimann et al. J Am Chem Soc. 2003 125(5):1203-20; Lawrence et al. Biophys Chem. 2002 Dec. 10; 101-102:375-85, herein specifically incorporated by reference.

The presence of alpha helical structure can also be predicted based on the amino acid sequence, e.g. as described by Phoenix et al. Curr Protein Pept Sci. 2002 April; 3(2):201-21; Mu\ddot{n}oz et al. Curr Opin Biotechnol. 1995 August; 6(4):382-6; Godzik et al. J Comput Aided Mol Des. 1993 August; 7(4):397-438; Viswanadhan et al. Biochemistry. 1991 Nov. 19; 30(46)11164-72; Gamier et al. Biochem Soc Symp. 1990; 57:11-24, herein specifically incorporated by reference.

Exemplary linkers include bacterial immunity proteins, fragments and derivatives thereof. Bacterial immunity proteins include colicin binding proteins, which can be obtained from various species of Enterobacteriaceae, including E. coli, Pseudomonas sp., Salmonella, sp., Yersinia, sp., Klebsiella sp., etc. Many of these proteins are plasmid encoded. The polypeptide sequences have a high degree of sequence identity to each other, e.g. an immunity protein of interest may have at least about 40%, at least about 50%, at least about 60%, at least about 70%, at least about 80%, at least about 90%, at least about 95% sequence identity to a polypeptide or more sequence identity at the amino acid level to a polypeptide sequence set forth in SEQ ID NO:1-15.

Immunity proteins can also be characterized by their structure. The proteins adopt a distorted, antiparallel four-helical structure with an all α-helical topology (see Ferguson et al. (1999) JMB 286:1597-1608, herein specifically incorporated by reference); lack disulphide bonds and prosthetic groups and may lack cis-Xaa prolyl peptide bonds in the native state.

In certain embodiments, the linker of the present invention is a polypeptide of from about 55 to about 90 amino acids in length, having at least about 90% or at least about 95% sequence identity to any one of SEQ ID NO:1-SEQ ID NO:15.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In certain embodiments, the linker of the present invention is a polypeptide of from about 55 to about 90 amino acids in length, which will fold into a thermodynamically stable structure from a linear form in less than about 10 seconds as determined by optimized in vitro refolding reactions, for example as described in the Examples.

In certain embodiments, the linker of the present invention is a polypeptide of from about 55 to about 90 amino acids in length, having 4 a helices in a distorted, antiparallel four-helical structure, and lacking disulphide bonds.

The fast-folding linker is joined at the amino terminus and at the carboxy terminus through peptide bonds to a first polypeptide and a second polypeptide. The first and second polypeptides are heterologous to the linker. As used herein, the term “heterologous” is intended to mean a polypeptide sequence that is not normally joined to the linker, e.g. in a native state. The first and the second polypeptide may be from a species other than a bacterial species. The first and the second polypeptide may be from the same or from a different protein.

Immunity Proteins

| Sequence | | | | |
|---|---|---|---|---|
| identifier | Organism | Gene | Genbank | SEQUENCE |
| SEQ ID NO: 1 | E. coli | Im9 | CAA33863 | ELKHSISDYTEAEFLQLVTTICNADTSSEEELVKLVTHFEEMT |
| | | | | EHPSGSDLIYYPKEGDDDSPSGIVNTVKQWRAANGKSGFKQG |
| SEQ ID NO: 2 | E. coli | Im6 | X15856 | GLKLHINWFDKRTEEFKGGEYSKDFGDDGSVIERLGMPFKDNI |
| | | | | NNGWFDVIAEWVPLLQPYFNHQIDISDNEYFVSFDYHDGDW |
| SEQ ID NO: 3 | E. coli | Im5 | X15857 | KLSPKAAIEVCNEAAKKGLWILGIDGGHWLNPGFRIDSSASWT |
| | | | | YDMPEEYKSKIPENNRLAIENIKDDIENGYTAFIITLKM |
| SEQ ID NO: 4 | E. coli | immHu194 | | ELKHSISDYTEAEFLEFVKKICRAEGATEEDDNKLVREFERLT |
| | | | | EHPDGSDLIYYPRDDREDSPEGIVKEIKEWRAANGKPGFKQG |
| SEQ ID NO: 5 | E. coli | Im7 | 1AYI | ELKNSISDYTEAEFVQLLKEIEKENVAATDDVLDVLLEHFVKI |
| | | | | TEHPDGTDLIYYPSDNRDDSPEGIVKEIKEWRAANGKPGFKQG |
| SEQ ID NO: 6 | Y. pestis | | | KFIQDVEENKMELKEKYEDYTEHEFLEFIRNICEVNTDSQSLH |
| | | | | SSWVRHFTKITEHPSGSDLIYYPEDGADDSPEGILELVKKWRA |
| | | | | ENGKPGFKK |
| SEQ ID NO: 7 | E. coli | immE8 | AAA23074 | ELKNSISDYTETEFKKIIEDIINCEGDEKKQDDNLEHFISVTE |
| | | | | HPSGSDLIYYPEGNNDGSPEAVIKEIKEWRAANGKSGFKQG |
| SEQ ID NO: 8 | Photorhabdus | | CAE14186 | KLNKKLEDYTEAEFLEFARKVCNADYATEDEANVAVQDFIRLS |
| | luminescens | | | EHPDGTDILFYPSSGQDDSPEGIVKQIKEWRAKSGKPGFKK |
| SEQ ID NO: 9 | Klebsiella | kbi | NP₋068717 | ANKTLADYTEQEFIEFIEKIKKADFATESEHDEAIYEFSQLTE |
| | pneumoniae | | | HPDGWDLIYHPQAGADNSPAGVVKTVKEWRAANGKPGFKKS |
| SEQ ID NO: 10 | Yersinia | pyocin S2 | CAH19391 | EDKSICDYTESEFLELVKELFNVEKTTEEEDINNLIEFKRLCE |
| | pseudotuber- | immunity | | HPAGSDLIFYPDNNREDSPEGVVKEVKKWRAENGKPGFKK |
| | culosis | protein | | |
| SEQ ID NO: 11 | Pseudomonas | pyocin S1 | BAA02202 | KSKISEYTEKEFLEFVEDIYTNNKKKFPTEESHIQAVLEFKKL |
| | aeruginosa | immunity | | TEHPSGSDLLYYPNENREDSPAGVVKEVKEWRASKGLPGFKAG |
| | | protein | | |
| SEQ ID NO: 12 | Pseudomonas | pyocin AP41 | BAA02197 | DIKNNLSDYTESEFLEIIEEFFKNKSGLKGSELEKRMDKLVKH |
| | aeruginosa | immunity | | FEEVTSHPRKSGVIFHPKPGFETPEGIVKEVKEWRAANGLPGF |
| | | protein | | KAG |
| SEQ ID NO: 13 | Salmonella | bacteriocin | YP₋152132 | KLKENISDYTESEFIDFLRVIFSENESDTDETLDPLLEYFEKI |
| | enterica | immunity | | TEYPGGTDLIYYPETESDGTPEGILNIIKEWRESQGLPCFKKS |
| | | protein | | K |
| SEQ ID NO: 14 | Pseudomonas | Pyocin S-type | AAN66929 | SEKTKLSDYTENEFLALIIEIHRANLEEPDHVLGGLLDHFSKI |
| | putida | immunity | | TEHPSGYDLLYRPNPKENGKPEKVLEIVKQWPLANGKDGFKPS |
| | | protein | | |
| SEQ ID NO: 15 | Salmonella | bacteriocin | YP₋152133 | ELKNNLEDYTEDEFIEFLNNFFEPPEELTGDELSKFIDNLLRH |
| | enterica | immunity | | FNKITQHPDGGDLIFYPSEEREDSPEGVIEELKRWRKSQRLPC |
| | | protein | | FKENK |

For use in the subject methods, native immunity proteins, for example as set forth in SEQ ID NO:1 to SEQ ID NO:15, or variants thereof may be used, where variants may comprise amino acid deletions, insertions or substitutions. Peptides of interest as linkers include fragments of at least about 45 contiguous amino acids, more usually at least about 50 contiguous amino acids, and may comprise 55 or more amino acids, up to the provided peptide. Deletions may extend from the amino terminus or the carboxy terminus of the protein, and may delete about 1, about 2, about 5, about 10, about 15 or more amino acids from either or both termini.

Substitutions or insertions may be made of 1, 2, 3, 4, 5, or more amino acids, where the substitutions may be conservative or non-conservative, so long as the fast folding nature of the protein is not changed. Typically, such substitutions may occur in the polypeptide loops connecting the secondary structural motifs (such as alpha-helical coils) and may introduce, for example, short polypeptides recognized for purification purposes. Scanning mutations that systematically introduce alanine, or other residues, may be used to determine key amino acids. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Optionally the linker peptide will be joined at one or both of the amino terminus and carboxy terminus with a short flexible linker, e.g. comprising at least about 2, 3, 4 or more glycine, serine and/or alanine residues. One such linker comprises the motif (GGGGS), and may be present in one or more copies.

Modifications of interest that do not alter primary sequence include chemical derivatization of polypeptides, e.g., acylation, pegylation, acetylation, or carboxylation. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. For examples, the backbone of the peptide may be cyclized to enhance stability (see Friedler et al. (2000) J. Biol. Chem. 275:23783-23789). Analogs of such polypeptides include those containing residues other than naturally occurring L-amino acids, e.g. D-amino acids or non-naturally occurring synthetic amino acids.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

### Fusion Protein Constructs

A first polypeptide and a second polypeptide are joined by a linker as described above to form a fusion polypeptide. By “fused” or “operably linked” herein is meant that the polypeptides are linked together to form a continuous polypeptide chain. As outlined below, the fusion polypeptide (or fusion polynucleotide encoding the fusion polypeptide) can comprise further components as well, including multiple peptides at multiple loops, fusion partners, etc. The precise site at which the fusion is made is not critical; particular sites are well known and may be selected in order to optimize the biological activity, secretion or binding characteristics of the binding partner. The optimal site will be determined by routine experimentation.

The first and second polypeptide components, which are separated by the linker, each provide for a distinct functional entity, e.g. an immunoglobulin variable region domain, an immunoglobulin single chain variable region domain, a cytokine domain, e.g. GM-CSF, etc. Such functional entities will typically correspond to one or more polypeptide domains. As is known in the art, a protein domain is a substructure produced by any part of a polypeptide chain that can fold independently into a compact, stable structure. A domain usually contains between about 35 to about 350 amino acids, and it is the modular unit from which many larger proteins are constructed. The different domains of a protein are often associated with different functions. The smallest protein molecules contain only a single domain, whereas larger proteins can contain as many as several dozen domains. The central core of a domain can be constructed from α helices, from β sheets, or from various combinations of these two fundamental folding elements. The invention further provides nucleic acids encoding the fusion polypeptides of the invention. As will be appreciated by those in the art, due to the degeneracy of the genetic code, an extremely large number of nucleic acids may be made, all of which encode the fusion proteins of the present invention. Thus, having identified a particular amino acid sequence, those skilled in the art could make any number of different nucleic acids, by simply modifying the sequence of one or more codons in a way that does not change the amino acid sequence of the fusion protein.

Using the nucleic acids of the present invention that encode a fusion protein, a variety of expression constructs can be made. The expression constructs may be self-replicating extrachromosomal vectors or vectors which integrate into a host genome. Alternatively, for purposes of cell-free expression the construct may include those elements required for transcription and translation of the desired polypeptide, but may not include such elements as an origin of replication, selectable marker, etc. Cell-free constructs may be replicated in vitro, e.g. by PCR, and may comprise terminal sequences optimized for amplification reactions.

Generally, expression constructs include transcriptional and translational regulatory nucleic acid operably linked to the nucleic acid encoding the fusion protein. The term “control sequences” refers to DNA sequences necessary for the expression of an operably linked coding sequence in a particular expression system, e.g. mammalian cell, bacterial cell, cell-free synthesis, etc. The control sequences that are suitable for prokaryote systems, for example, include a promoter, optionally an operator sequence, and a ribosome binding site. Eukaryotic cell systems may utilize promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. Linking is accomplished by ligation or through amplification reactions. Synthetic oligonucleotide adaptors or linkers may be used for linking sequences in accordance with conventional practice.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention. In a preferred embodiment, the promoters are strong promoters, allowing high expression in in vitro expression systems, such as the T7 promoter.

In addition, the expression construct may comprise additional elements. For example, the expression vector may have one or two replication systems, thus allowing it to be maintained in organisms, for example in mammalian or insect cells for expression and in a procaryotic host for cloning and amplification. In addition the expression construct may contain a selectable marker gene to allow the selection of transformed host cells. Selection genes are well known in the art and will vary with the host cell used.

In some embodiments of the invention, one polypeptide of the fusion protein is a cytokine. The term “cytokine” is a generic term for proteins released by one cell population which act on another cell as intercellular mediators. Examples of such cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen, OB protein; tumor necrosis factor-.alpha. and -.beta.; mullerian-inhibiting substance; mouse gonadotropin-associated peptide; inhibin; activin; vascular endothelial growth factor; integrin; thrombopoietin (TPO); nerve growth factors such as NGF-.beta.; platelet-growth factor; transforming growth factors (TGFs) such as TGF-.alpha. and TGF-.beta.; insulin-like growth factor-I and -II; erythropoietin (EPO); osteoinductive factors; interferons such as interferon-.alpha., -.beta. and -.gamma.; colony stimulatingfactors (CSFs) such as macrophage-CSF(M-CS F); granulocyte-macrophage-CSF(GM-CSF); and granulocyte-CSF (G-CSF); interleukins (ILs) such as IL-1, IL-1.alpha., IL-2, IL-3, IL-4, IL-5, IL-6, IL-7, IL-8, IL-9, IL-11, IL-12; and other polypeptide factors including leukemia inhibitory factor (LIF) and kit ligand (KL). As used herein, the term cytokine includes proteins from natural sources or from recombinant cell culture and biologically active equivalents of the native sequence cytokines.

Cytokines may be joined through a linker of the invention to antigens, e.g. for immunization purposes, where antigens include a variety of viral, bacterial, protozoan, etc. proteins and fragments thereof. Antigens may also include allergens. Antigens of interest also include tumor antigens, e.g. prostate specific antigen, etc.

### Immunoglobulin Domains

Generally, as the term is utilized in the specification, “immunoglobulin” or “immunoglobulin domain” is intended to include all types of immunoglobulins (IgG, IgM, IgA, IgE, IgD, etc.), from all sources (e.g., human, rodent, rabbit, cow, sheep, pig, dog, other mammal, chicken, turkey, emu, other avians, etc.). Immunoglobulins and variants thereof are known and many have been prepared in recombinant cell culture. For example, see U.S. Pat. No. 4,745,055; EP 256,654., Faulkner et al., Nature 298:286 (1982); EP 120,694; EP 125,023., Morrison, J. Immun. 123:793 (1979); Kohler et al., P.N.A.S. USA 77:2197 (1980); Raso et al., Cancer Res. 41:2073 (1981); Morrison et al., Ann Rev. Immunol. 2:239(1984); Morrison, Science 229:1202 (1985); Morrison et al., P.N.A.S. USA 81:6851 (1984); EP 255,694; EP 266,663; and WO 88/03559.

Immunoglobulin binding fragments may be produced by genetic engineering, by immunization, cloning from myeloma cells, etc. Typically, antibody-producing cells are sensitized to the desired antigen or immunogen. The mRNA isolated from the immune spleen cells or hybridomas is used as a template to make cDNA, from which the desired domain or domains is isolated. Chimeric antibodies may be made by recombinant means by combining the murine variable light and heavy chain regions (VK and VH), obtained from a murine (or other animal-derived) hybridoma clone, with the human constant light and heavy chain regions, in order to produce an antibody with predominantly human domains. Humanized antibodies are engineered to contain even more human-like immunoglobulin domains, and incorporate only the complementarity-determining regions of the animal-derived antibody. Immunoglobulin fragments comprising the epitope binding site (e.g., Fab′, F(ab′)₂, or other fragments) may comprise first polypeptide in a fusion protein of the present invention. For instance “scFv” domains may be produced by linking a variable light chain region to a variable heavy chain region via a peptide linker (e.g., poly-glycine or another sequence which does not form an alpha helix or beta sheet motif). Recombinant Fvs in which V_{H} and V_{L} are connected by a peptide linker are typically stable, see, for example, Huston et al., Proc. Natl. Acad, Sci. USA 85:5879-5883 (1988) and Bird et al., Science 242:423-426 (1988), both fully incorporated herein, by reference. Improved Fv's have been also been made which comprise stabilizing disulfide bonds between the V_{H} and V_{L} regions, as described in U.S. Pat. No. 6,147,203, incorporated fully herein by reference.

DNA encoding immunoglobulin light or heavy chain constant regions is known or readily available from cDNA libraries or is synthesized. See for example, Adams et al., Biochemistry 19:2711-2719 (1980); Gough et al., Biochemistry 19:2702-2710 (1980); Dolby et al; P.N.A.S. USA, 77:6027-6031 (1980); Rice et al P.N.A.S USA 79:7862-7865 (1982); Falkner et al; Nature 298:286-288 (1982); and Morrison et al; Ann. Rev. Immunol. 2:239-256 (1984).

DNA sequences encoding other desired polypeptides, e.g. cytokines, etc. which are known or readily available from cDNA libraries are suitable in the practice of this invention.

Chimeric polypeptides constructed from a polypeptide sequence linked to an appropriate immunoglobulin constant domain sequence are known in the art. Those reported in the literature include fusions of the T cell receptor (Gascoigne et al., Proc. Nat. Acad. Sci. USA 84: 2936-2940 (1987)); CD4 (Capon et al., Nature 337: 525-531 (1989); Traunecker et al., Nature 339: 68-70 (1989); Zettlmeissl et al., DNA Cell Biol. USA 9: 347-353 (1990); Byrn et al, Nature 344: 667-670 (1990)); L-selectin (homing receptor) ((Watson et al., J. Cell. Biol. 110:2221-2229 (1990); Watson et al, Nature 349:164-167(1991)); CD44 (Aruffo et al., Cell 61:1303-1313(1990)); CD28 and B7 (Lisley et al., J. Exp. Med. 173: 721-730 (1991)); CTLA4 (Lisley et al., J. Exp. Med. 174: 561-569 (1991)); CD22 (Stamenkovic et al., Cell 66:1133-1144 (1991)); TNF receptor (Ashkenazi et al, Proc. Natl. Acad. Sci. USA 88: 10535-10539 (1991); Lesslauer et al., Eur. J. Immunol 27: 2883-2886 (1991); Peppel et al., J. Exp. Med. 174:1483-1489 (1991)); NP receptors (Bennett et al., J. Biol. Chem. 266:23060-23067 (1991)); and IgE receptor α (Ridgway et al., J. Cell. Biol. 115:abstr. 1448 (1991.

The present invention provides for an improved chimeric composition, where the two polypeptides are joined through a linker of defined tertiary structure. One chimera design combines the binding region(s) of a protein of interest, through a linker of the invention, to the hinge and Fc regions of an immunoglobulin heavy chain. Typically, in such fusions the encoded chimeric polypeptide will retain at least functionally active hinge, CH2 and CH3 domains of the constant region of an immunoglobulin heavy chain. Fusions are also made to the C-terminus of the Fc portion of a constant domain, or immediately N-terminal to the CH1 of the heavy chain or the corresponding region of the light chain. The precise site at which the fusion is made is not critical; particular sites are well known and may be selected in order to optimize the biological activity, secretion or binding characteristics of the chimeras. In some embodiments, the chimeras are assembled as monomers, or hetero- or homo-multimers, and particularly as dimers or tetramers, essentially as illustrated in WO 91/08298.

Although the presence of an immunoglobulin light chain is not required, an immunoglobulin light chain might be present either covalently associated or directly fused to the polypeptide.

### Cell-Free Synthesis

In some embodiments of the invention, the fusion protein is produced by cell-free, or in vitro synthesis, in a reaction mix comprising biological extracts and/or defined reagents. The reaction mix will comprise a template for production of the macromolecule, e.g. DNA, mRNA, etc.; monomers for the macromolecule to be synthesized, e.g. amino acids, nucleotides, etc., and such co-factors, enzymes and other reagents that are necessary for the synthesis, e.g. ribosomes, tRNA, polymerases, transcriptional factors, etc. Such synthetic reaction systems are well-known in the art, and have been described in the literature. A number of reaction chemistries for polypeptide synthesis can be used in the methods of the invention. For example, reaction chemistries are described in U.S. Pat. No. 6,337,191, issued Jan. 8, 2002, and U.S. Pat. No. 6,168,931, issued Jan. 2, 2001, herein incorporated by reference.

In one embodiment of the invention, the reaction chemistry is as described in international patent application WO 2004/016778, herein incorporated by reference. The activation of the respiratory chain and oxidative phosphorylation is evidenced by an increase of polypeptide synthesis in the presence of O₂. In reactions where oxidative phosphorylation is activated, the overall polypeptide synthesis in presence of O₂ is reduced by at least about 40% in the presence of a specific electron transport chain inhibitor, such as HQNO, or in the absence of O₂. Improved yield is obtained by a combination of factors, including the use of biological extracts derived from bacteria grown on a glucose containing medium; an absence of polyethylene glycol; and optimized magnesium concentration. This provides for a homeostatic system, in which synthesis can occur even in the absence of secondary energy sources.

The template for cell-free protein synthesis can be either mRNA or DNA. Translation of stabilized mRNA or combined transcription and translation converts stored information into protein. The combined system, generally utilized with a bacterial extract, e.g. an Enterobacteriaceae extract, including E. coli, Erwinia, Pseudomonas, Salmonella, etc., continuously generates mRNA from a DNA template with a recognizable promoter. Either endogenous RNA polymerase is used, or an exogenous phage RNA polymerase, typically T7 or SP6, is added directly to the reaction mixture. Alternatively, mRNA can be continually amplified by inserting the message into a template for QB replicase, an RNA dependent RNA polymerase. Purified mRNA is generally stabilized by chemical modification before it is added to the reaction mixture. Nucleases can be removed from extracts to help stabilize mRNA levels. The template can encode for any particular gene of interest.

Other salts, particularly those that are biologically relevant, such as manganese, may also be added. Potassium is generally added between 50-250 mM and ammonium between 0-100 mM. The pH of the reaction is generally between pH 6 and pH 9. The temperature of the reaction is generally between 20° C. and 40° C. These ranges may be extended.

Metabolic inhibitors to undesirable enzymatic activity may be added to the reaction mixture. Alternatively, enzymes or factors that are responsible for undesirable activity may be removed directly from the extract or the gene encoding the undesirable enzyme may be inactivated or deleted from the chromosome.

Vesicles, either purified from the host organism or synthetic, may also be added to the system. These may be used to enhance protein synthesis and folding. This cytomim technology has been shown to activate processes that utilize membrane vesicles containing respiratory chain components for the activation of oxidative phosphorylation.

Synthetic systems of interest include the replication of DNA, which may include amplification of the DNA, the transcription of RNA from DNA or RNA templates, the translation of RNA into polypeptides, and the synthesis of complex carbohydrates from simple sugars.

The reactions may be large scale, small scale, or may be multiplexed to perform a plurality of simultaneous syntheses. Additional reagents may be introduced to prolong the period of time for active synthesis. Synthesized product is usually accumulated in the reactor, and then is isolated and purified according to the usual methods for protein purification after completion of the system operation.

Of particular interest is the translation of mRNA to produce proteins, which translation may be coupled to in vitro synthesis of mRNA from a DNA template. Such a cell-free system will contain all factors required for the translation of mRNA, for example ribosomes, amino acids, tRNAs, aminoacyl synthetases, elongation factors and initiation factors. Cell-free systems known in the art include E. coil extracts, etc., which can be prepared using a variety of methods. Methods for producing active extracts are known in the art, for example they may be found in Pratt (1984), Coupled transcription-translation in prokaryotic cell-free systems, p. 179-209, in Hames, B. D. and Higgins, S. J. (ed.), Transcription and Translation: A Practical Approach, IRL Press, New York. Kudlicki at al. (1992) Anal Biochem 206(2):389-93 modify the S30 E. coli cell-free extract by collecting the ribosome fraction from the S30 by ultracentrifugation. Zawada and Swartz Biotechnol Bioeng, 2006. 94(4): p. 618-24 teach a modified procedure for extract preparation.

In addition to the above components such as cell-free extract, genetic template, and amino acids, materials specifically required for protein synthesis may be added to the reaction. These materials include salts, polymeric compounds, cyclic AMP, inhibitors for protein or nucleic acid degrading enzymes, inhibitors or regulators of protein synthesis, oxidation/reduction adjusters, non-denaturing surfactants, buffer components, spermine, spermidine, etc.

The salts preferably include potassium, magnesium, and ammonium salts of acetic acid or glutamic acid, and some of these may have an alternative amino acid as a counter anion. The polymeric compounds may be polyethylene glycol, dextran, diethyl aminoethyl dextran, quaternary aminoethyl and aminoethyl dextran, etc. The oxidation/reduction adjuster may be dithiothreitol, ascorbic acid, cysteine, glutathione and/or their oxides. Also, a non-denaturing surfactant such as Brij-35 may be used at a concentration of 0-0.5 M. Spermine and spermidine may be used for improving protein synthetic ability, and cAMP may be used as a gene expression regulator.

When changing the concentration of a particular component of the reaction medium, that of another component may be changed accordingly. For example, the concentrations of several components such as nucleotides and energy source compounds may be simultaneously controlled in accordance with the change in those of other components. Also, the concentration levels of components in the reactor may be varied over time.

Preferably, the reaction is maintained in the range of pH 5-10 and a temperature of 20°-50° C., and more preferably, in the range of pH 6-9 and a temperature of 25°-40° C.

The amount of protein produced in a translation reaction can be measured in various fashions. One method relies on the availability of an assay which measures the activity of the particular protein being translated. Examples of assays for measuring protein activity are a luciferase assay system, and a chloramphenical acetyl transferase assay system. These assays measure the amount of functionally active protein produced from the translation reaction. Activity assays will not measure full-length protein that is inactive due to improper protein folding or lack of other post translational modifications necessary for protein activity.

Another method of measuring the amount of protein produced in a combined in vitro transcription and translation reactions is to perform the reactions using a known quantity of radiolabeled amino acid such as ³⁵S-methionine or ¹⁴C-leucine and subsequently measuring the amount of radiolabeled amino acid incorporated into the newly translated protein. Incorporation assays will measure the amount of radiolabeled amino acids in all proteins produced in an in vitro translation, reaction including truncated protein products. The radiolabeled protein may be further separated on a protein gel, and by autoradiography confirmed that the product is the proper size and that secondary protein products have not been produced.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the reagents, cells, constructs, and methodologies that are described in the publications, and which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Experimental

### Example 1

The 38C13 mouse B-cell lymphoma Id scFv protein is fused to mouse GM-CSF connected by a normal GGGGS linker or an Im9 linker, giving rise to an immunoglobulin construct. The fusion proteins with and without the Im9 linker were expressed in the cell-free protein synthesis system. Their cell-free expression yields and purification are compared. The result shows that the fusion structure with the Im9 linker has a higher soluble yield than the fusion construct without it. Also, the Im9 linker improves the polypeptide stability during purification.

### Construction of the Fusion Protein Expression Plasmids

First, the gene that encodes the fusion protein, GM-VL-VH, was constructed, which contains the variable regions of 38C13 Id protein and mouse GM-CSF. GM-CSF protein, which is located at the N-terminus of the fusion structure, is connected to the scFv domain through a five amino acid linker GGGGS. The GM-CSF is also extended at its N-terminus by the first five codons of CAT (E.coli chloramphenicol acetyl transferase), which is 5′ATGGAGAAAAAAATC3′. The GM-VL-VH construct is subcloned into the expression plasmid pK7(Yang, J. et al (2005) Biotechnol. Bioeng. 89: 503-511) yielding pK7catgmvlvh.

Second, the Im9 linker is inserted into the GM-VL-VH structure before the GGGGS linker, yielding a new fusion structure GM-Im9-VL-VH. Im9 is an E. coli immunity protein which contains 85 amino acids (Ferguson, N. et al (1999) J. Mol. Biol. 286: 1597-1608). The DNA sequence encoding the Im9 protein is designed to use the codons which are favored for protein expression in E. coil system, which is:

(SEQ ID (SEQ ID NO:16) 5′GAACTGAAACATAGCATCTCCGACTATACCGAAGCGGAGTTTTTACAGCTGGTGACC ACGATTTGCAACGCCGATACCAGCTCGGAAGAAGAGCTGGTGAAATTAGTGACGCATT TTGAAGAGATGACCGAGCATCCGAGCGGTTCCGATCTGATTTACTATCCGAAAGAGGG CGATGACGATAGCCCGAGTGGGATTGTTAACACCGTTAAACAGTGGCGTGCGGCCAA TGGTAAAAGCGGGTTTAAACAAGGG3′. To make this new linker, 6 synthesized DNA fragments are mixed for overlapping PCR (annealing temperature at 54° C.), resulting in a DNA encoding Im9-GGGGS and some additional amino acids which are identical to the N-terminal peptide of VL (the variable region of the light chain).

These DNA fragments include (SEQ ID NO:17) 5′ GAACTGA AACATA GCATCTC CGACTATACCGAAGC GGAGTTTT TACAGCTGGTG ACCACG ATTTGCAAC GCCGATACCAG3′, (SEQ ID NO:18) 5′CGGATGCT CGGTCATCTCT TCAA AATGCG TCACTAATTTCAC CAGCTCTTCTTCCGAG CTGGT ATCGGCG TTGCAAATC3′, (SEQ ID NO:19) 5′GAAGAGATG ACCGAGCATCCGAGCGGT TCCGATCTGAT TTACTATCCGAAAGAGGG CGATGACGA TAGCCCGAG3′, (SEQ ID NO:20) 5′GTTTAAACC CGCTTTT ACCATTGGCCG CACGCCACTGTTTAACGGTGTTAAC AATCCCACT CGGGCTAT CGTCATCGC3′, (SEQ ID NO:21) 5′CCAATGGTAAAAGCGGGT TTAAAC AAGGGGGTGGCGGTGG CAGCGA CATTGAGCT CACCCAGTCT3′, and (SEQ ID NO:22) 5′AGACTGGGTGAGCTCAATGTC′. Then the PCR amplified Im9 fragment is ligated to mouse GM-CSF through overlapping PCR again. GM-CSF is amplified with (SEQ ID NO:23) 5′ATATACATATGGAGAAAAAAATCGCACC3′ and (SEQ ID NO:24) 5′GTCGG AGATGCTA TGTTTC AGTTCA GAGCCACCTCCTCCTTTTTG3′ as primers and pK7catgm (Yang, J. et al (2004) Biotechnol. Prog. 20: 1689-1696) as template. The PCR amplified GM-CSF is mixed with the IM9 fragment and ten rounds of annealing and extension are conducted, followed by PCR with (SEQ ID NO:25) 5′ATATACATATGGAGAAAAAAATCGCACC3′ and (SEQ ID NO:26) 5′ AGACTGGGTGAGCTCAATGTC3′. Finally, the PCR amplified GM-Im9 fragment is digested by Nde I and Sac I, and ligated into Nde I/Sac I digested pK7catgmvlvh, yielding pK7catgmim9vlvh.

Finally, a His6-tag and the GGGGS sequence is ligated at the N-terminus of the fusion constructs after the first five amino acid sequence through PCR extension. The GM-VL-VH construct with N-terminal His6-tag is amplified with 5catNhisG4S, (SEQ ID NO:27) 5′ATATATACATATGGAGAAAAAAATCCATCACCACCATCATCACGGAGGAGGAGGTTC AGCACCCACCCGCTCACCC3′, and 3salvH, (SEQ ID NO:28) 5′TATATATGTCGACTTATTA TGAGGAGACGG TGACCGTGG3′, using pK7catgmvlvh as template. The amplified PCR fragment is digested with Nde I/Sal I and ligated with pK7 plasmid, yielding pK7cathisgmvlvh. Similarly, the GM-Im9-VL-VH with N-terminal His6-tag is amplified with 5catNhisG4S and 3salvH using pK7catgmim9vlvh as template. The amplified PCR fragment is digested with Nde I/Sal I and ligated with pK7 plasmid, yielding pK7cathisgmim9vlvh.

### Cell-Free Expression of Immunoglobulin Constructs

The cell-free expression of immunoglobulin constructs is carried out as described previously (Yang, J. et al (2005) Biotechnol. Bioeng. 89: 503-511). The fusion proteins, encoded by pK7catgmvlvh and pK7catgmim9vlvh, are expressed in 6 well tissue-culture plates (Falcon) when they are produced at 1 ml scale. The cell-free reaction is carried out at 30° C. for 4 hours. After the reaction, the soluble fraction is harvested after centrifugation at 14,000 g for 15 min. The total protein yield and soluble protein yield of GM-VL-VH and GM-Im9-VL-VH are calculated through the amount of radioactive leucine incorporated into the TCA-insoluble fraction.

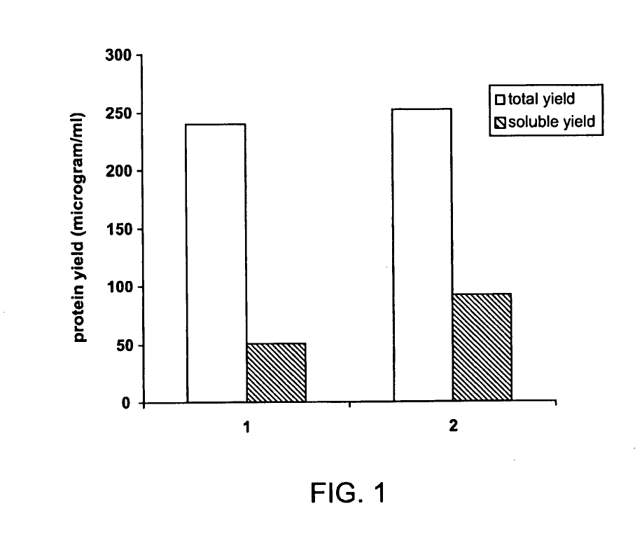

The results show that the construct with the Im9 linker has a higher soluble protein yield than the original construct (FIG. 1).

### Protein Purification

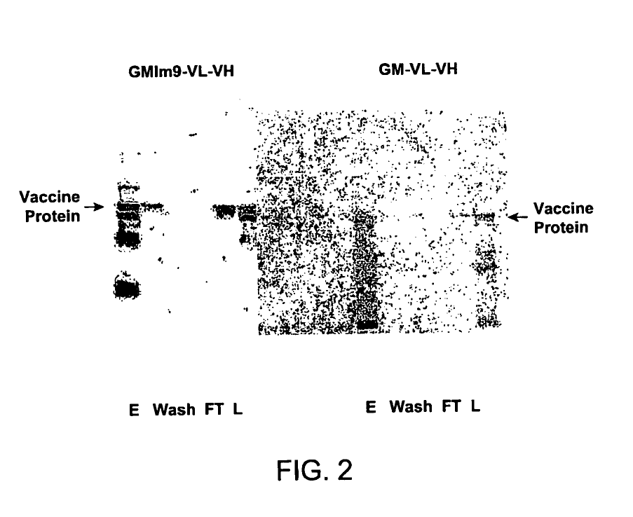

The cell-free expressed GM-VL-VH and GM-Im9-VL-VH are purified with a 1 ml HisTrap chelating column as previously described (Yang, J. et al (2004) Biotechnol. Prog. 20: 1689-1696). The purification samples are analyzed by SDS-PAGE and autoradiography as described before (Yang, J. et al (2005) Biotechnol. Bioeng. 89: 503-511). The purification results show that the fusion construct with the Im9 linker results in significantly more full length fusion protein and less truncated product than the original construct after purification (FIG. 2).

The linkers in fusion protein structures are frequently short, flexible peptides (George, R. A. and Heringa, J. (2003) Protein Eng. 15: 871-879). In contrast, this invention uses a whole protein as a linker to connect the two domains of the B cell immunoglobulin protein. This Im9 protein folds very quickly into a defined tertiary structure, therefore it will not interfere the folding of the two protein domains it connects. Another advantage of this long peptide linker is to separate the two domains of the fusion protein. Therefore, it will decrease the interference of the two protein domains during folding.

The improved protein folding of the new construct with the Im9 linker resulted in a higher soluble protein yield in the cell-free protein synthesis system. Another result of the improved folding is that the new construct is more stable during its production and purification. Comparing with the old construct with the GGGGS linker, the new fusion protein with the Im9 linker shows less degradation after purification.

What is claimed is:

1. A method for increased yield of a fusion protein, the method comprising:
separating a first polypeptide of said fusion protein and a second polypeptide of said fusion protein with a linker of defined tertiary structure and at least about 45 amino acids in length;
wherein the yield of full-length fusion protein is increased by at least 20%.

2. The method of claim 1, wherein the linker comprises at least 3 alpha helices.

3. The method of claim 2, wherein the linker is not more than 100 amino acids in length.

4. The method of claim 3, wherein the linker is a bacterial immunity protein or fragment thereof.

5. The method of claim 3, wherein the linker has at least 95% sequence identity with a polypeptide set forth in any one of SEQ ID NO:1-15.

6. The method according to claim 1, wherein said the fusion protein is synthesized in a cell-free reaction mixture comprising a bacterial cell extract, components of polypeptide and/or mRNA synthesis machinery; a template for transcription of the polypeptide; monomers for synthesis of the polypeptide; co-factors and enzymes necessary for translation.

7. The method of claim 6 wherein said synthesis also comprises transcription of mRNA from a DNA template.

8. A fusion protein comprising:
a first polypeptide and a second polypeptide, wherein said first polypeptide and said second polypeptide are joined by a linker of defined tertiary structure and at least about 45 amino acids in length.

9. The fusion protein of claim 8, wherein the linker comprises at least 3 alpha helices.

10. The fusion protein of claim 9, wherein the linker is not more than 100 amino acids in length.

11. The fusion protein of claim 10, wherein the linker is a bacterial immunity protein or fragment thereof.

12. The fusion protein of claim 11, wherein the linker has at least 95% sequence identity with a polypeptide set forth in any one of SEQ ID NO:1-15.

13. The fusion protein of claim 10, wherein said first polypeptide and said second polypeptide each provide for a distinct functional entity.

14. The fusion protein of claim 13, wherein said functional entity comprises at least one polypeptide domain.
